(19) United States
(12) Patent Application Publication
(10) Pub. No.: US 2006/0173374 A1
(43) Pub. Date: Aug. 3, 2006

(54) Electrically insulated surgical probing tool

(76) Inventors: Seth L. Neubardt, White Plains, NY (US); Sharonda Felton, Cordova, TN (US)

Correspondence Address: KRIEG DEVAULT LLP, ONE INDIANA SQUARE, SUITE 2800, INDIANAPOLIS, IN, 46204-2709, US

(21) Appl. No.: 11/047,357

(22) Filed: Jan. 31, 2005

(51) Int. Cl. A61B 5/05

(52) U.S. Cl. 600/547; 600/546

(57) ABSTRACT

A surgical tool for probing bone tissue includes an elongate member coupled to a handle assembly. The handle assembly is electrically coupled to a electrical signal source. The surgical tool includes an electrically conductive portion in communication with an un-insulated distal end of the elongate member, and insulated portion extending from the tip along the elongate member and handle assembly.

### BACKGROUND

Monitoring of the location of neural elements can reduce the likelihood of neural damage while accessing structures, such as bone or muscle, near the nerve. Surgical tools exist which provide an electrical potential to allow for detection of neural element proximity by visibly noting a patient's limb motor reaction when the neural element is stimulated by electrical current. A refinement of this detection method uses a plurality of electric signals; location of the neural element is determined by comparing these electrical signals to a calibration electrode, thereby eliminating the need for physical monitoring of a patient's limb.

### SUMMARY

The present apparatus, kit and method provides the surgeon the ability to probe bone tissue and monitor proximity of neural elements while enhancing the ability to control and manipulate the surgical tool during the procedure. The device comprises a surgical tool for insertion into bone tissue while delivering an electrical signal to monitor a proximity of neural elements to the inserted end of the tool.

In one embodiment, the device includes an elongate member with an electrically conductive portion and an insertion portion near its distal end, an insulated surface area between its distal and proximal ends and a conductive path between the electrically conductive portion near its distal end and a place near the proximal end. The device has a handle assembly with continuously curved surfaces at interfaces with the user's hand at a gripping portion having a major dimension at least 50% greater than its minor dimension as measured orthogonally to a longitudinal axis of the elongate member and orthogonally to one another. The handle assembly is attached near the proximal end of the elongate member and has an electrically insulated surface area and an electrically conductive area internal to the electrically insulated surface area.

In another embodiment, the device includes an elongate member with an electrically conductive portion and an insertion portion near its distal end, an insulated surface area between its distal and proximal ends and a conductive path between the electrically conductive portion near its distal end and a place near the proximal end. The handle assembly is attached near the proximal end of the elongate member and has an electrically insulated surface area and an electrically conductive area internal to the electrically insulated surface area. The handle assembly has a gripping portion with a major dimension that is at least 50% greater than a minor dimension as measured orthogonally to a longitudinal axis of the elongate member and orthogonally to one another.

A further embodiment has an elongate member with an electrically conductive portion and an insertion portion near its distal end, an insulated surface area between its distal and proximal ends and a conductive path between the electrically conductive portion near its distal end and a place near the proximal end. The handle assembly is attached near the proximal end of the elongate member and has an electrically insulated surface area and an electrically conductive area internal to the electrically insulated surface area. The device has a handle assembly with continuously curved surfaces at interfaces with the user's hand and a major dimension that is at least 50% greater than a minor dimension as measured orthogonally to a longitudinal axis of the elongate member and orthogonally to one another.

An illustrated embodiment includes an elongate member with an electrically conductive portion and an insertion portion near its distal end, an insulated surface area between its distal and proximal ends and a conductive path between the electrically conductive portion near its distal end and a place near the proximal end. The elongate member also has a notch near the proximal end. The handle assembly is attached near the proximal end of the elongate member and has an electrically insulated surface area and an electrically conductive area internal to the electrically insulated surface area. The handle assembly also has an opening for receiving the proximal portion of the elongate member in an overlapping arrangement. The surgical tool also has a locking element rotatable around the elongate member from a position that retains the elongate member in the handle assembly to a position that allows removal of the elongate member from the handle assembly. The locking element can rotate to a position to engage the notch of the elongate member.

In another embodiment, the surgical tool has an elongate member with an electrically conductive portion and an insertion portion near its distal end, an insulated surface area between its distal and proximal ends and a conductive path between the electrically conductive portion near its distal end and a place near the proximal end. The elongate member also has a notch near the proximal end. The handle assembly is attached near the proximal end of the elongate member and has an electrically insulated surface area and an electrically conductive area internal to the electrically insulated surface area. The handle assembly also has an opening for receiving the proximal portion of the elongate member in an overlapping arrangement. The handle assembly further has continuously curved surfaces at interfaces with the user's hand and a major dimension that is at least 50% greater than a minor dimension as measured orthogonally to a longitudinal axis of the elongate member and orthogonally to one another. The surgical tool also has a locking element rotatable around the elongate member from a position that retains the elongate member in the handle assembly to a position that allows removal of the elongate member from the handle assembly. The locking element can rotate to a position to engage the notch of the elongate member.

In another embodiment, the surgical tool has an elongate member with an electrically conductive portion and an insertion portion near its distal end, an insulated surface area between its distal and proximal ends and a conductive path between the electrically conductive portion near its distal end and a place near the proximal end. The elongate member also has a notch near the proximal end. The handle assembly is attached near the proximal end of the elongate member and has an electrically insulated surface area and an electrically conductive area internal to the electrically insulated surface area. The handle assembly also has an opening for receiving the proximal portion of the elongate member in an overlapping arrangement. The handle assembly has a major dimension that is at least 50% greater than a minor dimension as measured orthogonally to a longitudinal axis of the elongate member and orthogonally to one another. The surgical tool also has a locking element rotatable around the elongate member from a position that retains the elongate member in the handle assembly to a position that allows removal of the elongate member. The locking element can rotate to a position to engage the notch of the elongate member from the handle assembly.

In another embodiment, the surgical tool has an elongate member with an electrically conductive portion and a cutting portion near its distal end, an insulated surface area between its distal and proximal ends and a conductive path between the electrically conductive portion near its distal end and a place near the proximal end. The elongate member also has a notch near the proximal end. The handle assembly is attached near the proximal end of the elongate member and has an electrically insulated surface area and an electrically conductive area internal to the electrically insulated surface area. The handle assembly also has an opening for receiving the proximal portion of the elongate member in an overlapping arrangement. The handle assembly further has continuously curved surfaces at interfaces with the user's hand and a major dimension that is at least 50% greater than a minor dimension. The surgical tool also has a locking element rotatable around the elongate member from a position that retains the elongate member in the handle assembly to a position that allows removal of the elongate member from the handle assembly. The locking element can rotate to a position to engage the notch of the elongate member.

In one embodiment, the elongate member is a probe member and the insertion end is a distal tip of the probe member. The probe member can be configured for use in cervical, thoracic, sacral, or lumbar spinal procedures, and may include a straight or non-straight configuration along all or a portion of its length.

In an embodiment, when attached, the connection between the handle assembly and elongate member is secure and entirely insulated. In another embodiment, the elongate member has an electrically conductive end portion at the proximal end. The conductive end portion fits inside an opening in the handle assembly. This connection allows for the entire electrically conductive end portion of the elongate member to be electrically insulated inside the handle assembly while providing an internal and removable electrical connection to an electrical signal source.

### BRIEF DESCRIPTION OF THE FIGURES

FIG. 1 is a view of the surgical field with an assembled perspective view of the surgical tool.

FIGS. 2A-D show a set of detachable elongate members for use with the handle assembly in FIG. 3.

FIG. 3 is a perspective view of the handle assembly.

FIG. 4 is a view from the distal end of the handle assembly.

FIG. 5 is a cross-section of the handle assembly through line 5-5 of FIG. 4.

FIG. 6 is a side elevational view of the handle assembly rotated 180 degrees from its FIG. 5 orientation.

FIG. 7 is section of the handle assembly through line 7-7 of FIG. 6.

FIG. 8A is a perspective view of the locking element of the surgical tool shown in FIG. 1.

FIG. 8B is a side elevational view of the locking element shown in FIG. 8A.

### DETAILED DESCRIPTION OF THE ILLUSTRATED EMBODIMENTS

While this device is susceptible of embodiment in many different forms, there is shown in the drawings, and will herein be described in detail, several specific embodiments, with the understanding that the present disclosure can be considered as an exemplification and is not intended to be limited to the embodiments illustrated.

The system, method and kit relates to surgical tools and more particularly to surgical tools used in determining the proximity of neural elements. The surgical tool includes an elongate member, such as a probe, and a handle assembly. In one embodiment, the elongate member is removably engageable to the handle assembly with a locking element, although embodiments without a locking element are also contemplated. The surgical tool is operable to deliver an electrical signal, such as a current, to a location in the patient's body to monitor proximity of neural elements to the inserted end of the tool. A lead connects the handle assembly to an electrical signal source, which may comprise a porion of a nerve monitoring system such as the NIM-Spine™ System marketed by Medtronic, Inc. or any other suitable nerve monitoring system. Another lead can be used to ground the circuit. The surgical tool, when assembled, is completely insulated except for the insertion end to prevent shunting of the electrical signal to adjacent tissue or instruments.

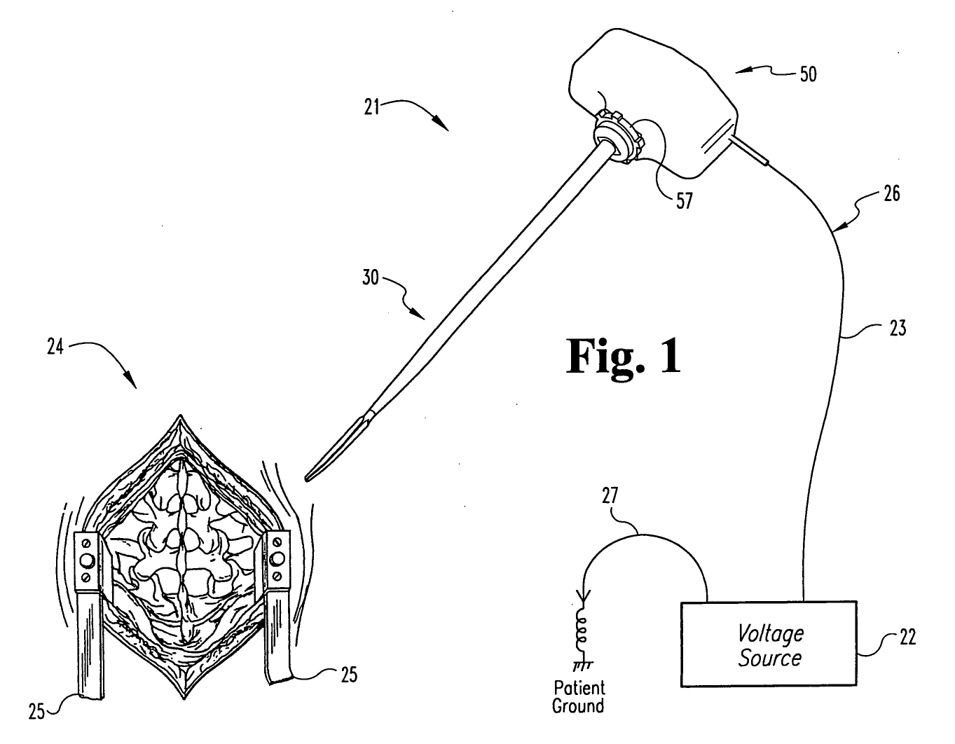

FIG. 1 is a view of the surgical field 24 with an assembled perspective view of the surgical tool 21. A midline incision has been made in the lumbar region of interest. Retractor arms 25 keep the surgical field 24 open sufficiently to allow the desired use and positioning of the surgical tool 21. Surgical tool 21 comprises an elongate member 30 and a handle assembly 50. A voltage source 22 is coupled to surgical tool 21 via a conductive path having a first reference 23 coupled to surgical tool 21 and a second reference 27 coupled to a patient (not shown). The second reference 27 is a ground, and can be connected to patient muscle tissue adjacent the surgical field. The ground can also be established by using a conventional surgical grounding pad that has been affixed to the patient. Although the posterior lumbar spinal region is shown for the purpose of illustration, the surgical tool is not limited in application to a posterior approach or the lumbar region, as will be appreciated by those skilled in the art.

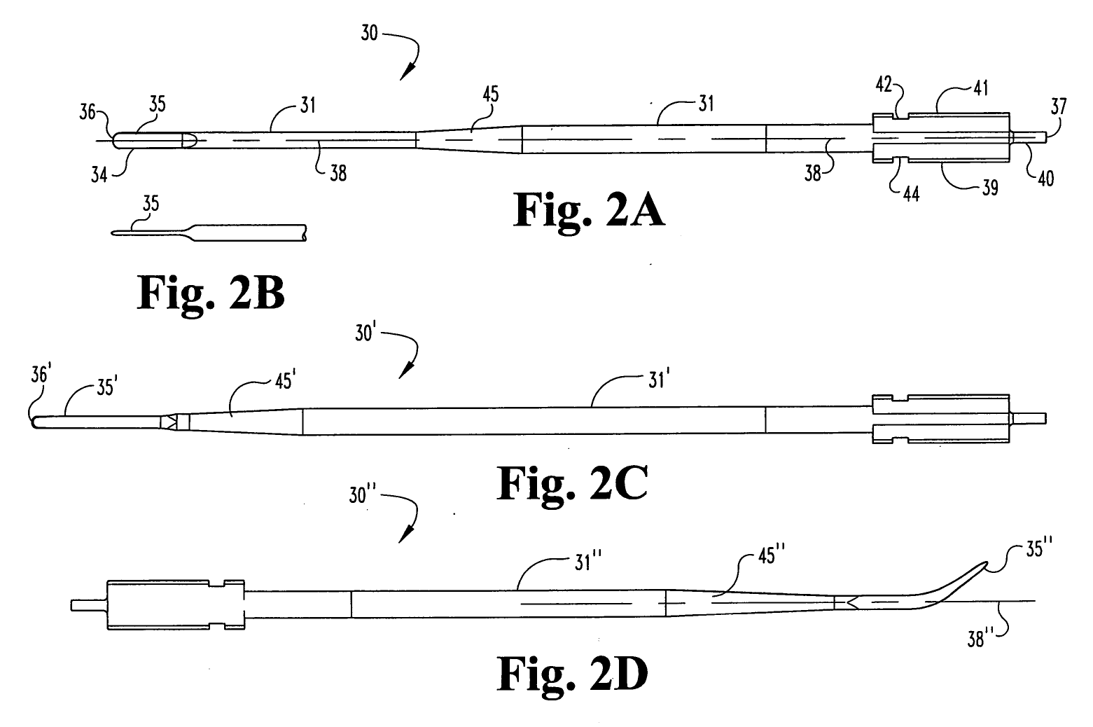

Elongate member 30 is in the form of a probe with a distal probing end insertable in bone tissue or in a hole in bone tissue to probe the hole and assist in hole formation. FIGS. 2A-D show various embodiments for elongate member 30 capable of being attached to handle assembly 50. Elongate member 30 comprises an exposed or no-insulated electrically conductive insertion portion 34 extending along a longitudinal axis 38 forming a probe end 35 adjacent to a distal end 36. An insulated shaft portion 31 that provides an insulated, conductive path between distal end 36 and a proximal end 37. An attaching portion 39 near proximal end 37 includes a proximally extending stem 40 extending proximally from a barrel portion 41. A first notch 42 and an opposite second notch 44 are formed in barrel portion 41 to receive a locking element to couple elongate member 30 to a handle assembly, as discussed further below.

FIG. 2A shows a straight elongate member 30 including shaft portion 31 wih an intermediate tapered portion 45. The straight elongate member 30 has an exposed, non-insulated probe end 35 near the distal end 36. Probe end 35 can be distally tapered and in a linear configuration to facilitate placement into the bone tissue. As shown in FIG. 2B, probe end 35 is flattened in at least one direction relative to the longitudinal axis 38.

FIG. 2C shows an embodiment elongate member 30′ suited for use in the lumbar region of the spine. Elongate member 30′ had an insulated shaft portion 31 and includes an exposed probe end 35′ near the distal end 36′ that includes a uniform thickness extending to a rounded or bullet shaped distal tip. Elongate member 30′ further includes a tapered shaft portion 45′ that is positioned more distally than intermediate tapered shaft portion 45 of elongate member 30. FIG. 2D shows a thoracic elongate member 30″ that includes an insulated shaft portion 31″, a tapered portion 45″, and a distal probe end 35″. Probe end 35″ includes a distally tapered outer surface profile extending to a rounded or bullet shaped distal tip. Probe end 35″ includes an angled or curved configuration so that it extends transversely to longitudinal axis 38″ of shaft portion 31″. Other forms for the elongate member are also contemplated, including those with curved portions.

With any of these or another embodiment elongate member 30 attached, the surgical tool 21 may be employed to probe bone tissue and deliver an electrical signal to detect the presence and proximity of neural elements. The probe end can be employed for forming, shifting, piercing, stabbing, penetrating, dissecting, resecting or otherwise perform functions relative to the bone tissue.

Elongate member 30 may be made of stainless surgical steel or other suitable conductive material of sufficient strength. Elongate member 30 can be constructed from a single piece of suitable conductive material or could be constructed from more than one piece of suitable conductive material. Barrel portion 41 and the remainder of the elongate member 30 could be separate pieces. The insulated surface area between the distal and proximal ends 37 may be achieved through the use of a coating, e.g. polyamide coating or through other means, such as an overlaying sleeve of foam or other material. The insulated surface area ensures the electrical signal is directed to the target area and is not shunted to surrounding, unintended, tissue or surgical instruments.

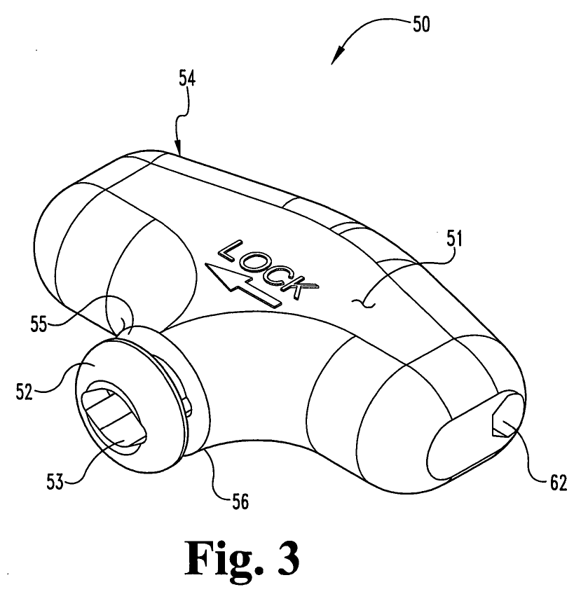

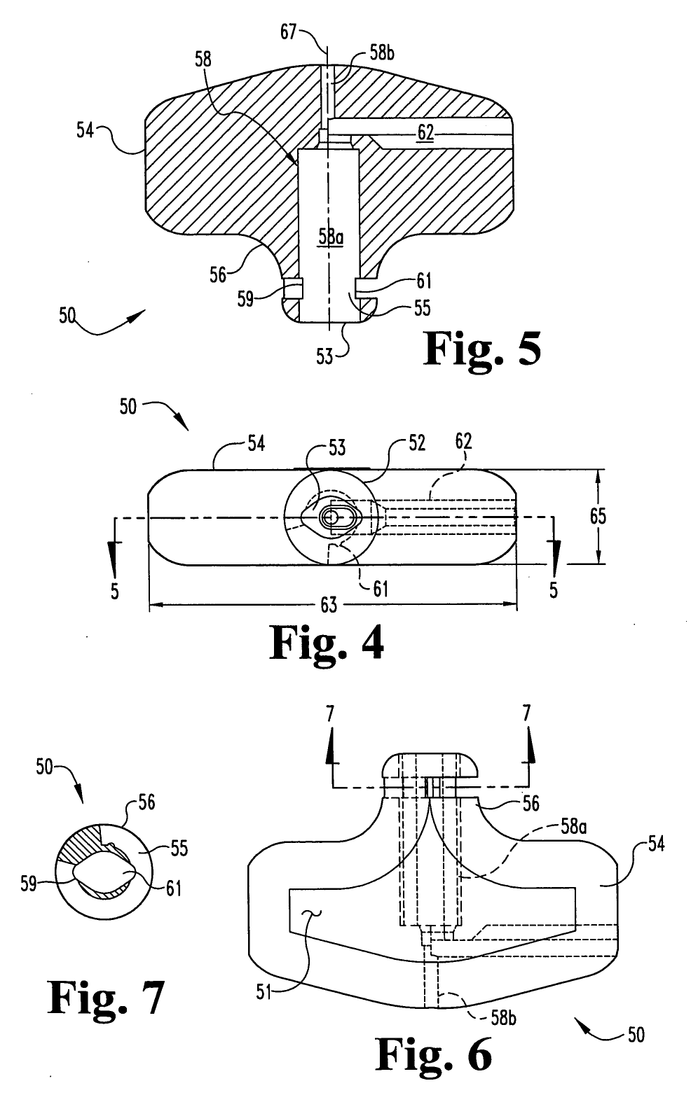

Handle assembly 50 is shown in FIGS. 3, 4, 5, and 6. Handle assembly 50 comprises a handle body 54 with an electrically insulated surface area 51 and an electrically conductive area internal to handle body 54. Handle body 54 further includes a distally facing opening 53 in a distally extending neck portion 56. Neck portion 56 includes a channel 55 that receives a locking element 57 (FIGS. 1 and 7-8.). An elongate member passage 58 extends axially through at least a portion of handle body 54. A relaying chamber 62 extends transversely to passage 58 and is sized and configured to receive an electrical lead 23.

Body 54 of handle assembly 50 has a major dimension 63 and a minor dimension 65. The major and minor dimensions 63, 65 are measured orthogonally to one another and orthogonally to an extension of longitudinal axis 38 axially through handle body 54 when elongate member 30 is assembled thereto. In one embodiment, the major dimension is at least 50% greater than the minor dimension. The proximal end of body 54 includes continuously curved surfaces at its interface with the user's hand. This enables a user to have a secure and comfortable grasp on the handle assembly 50. Furthermore, chamber 62, which receives lead 26, extends along the major dimension to position lead 26 away from the gripping surfaces of body 54, preventing lead 26 from interfering with gripping and control of surgical tool 21. The shape of handle body 54 provides body 54 with a gripping portion that anatomically accommodates the hand of the surgeon or other attendant, and facilitates manipulation and control of surgical tool 21 with handle assembly 50.

Opening 53 leads into elongate member passage 58, which extends axially along central axis 67 through the interior of handle body 54. Elongate member passage 58 has the same cross-section shape as barrel portion 41 of elongate member 30, and receives barrel portion 41 when elongate member 30 and handle assembly 50 are joined together. In the present embodiment, opening 53 has an oblong shape so that elongate member 30 is non-rotatably received in handle body 54.

When assembled, attaching portion 39 of elongate member 30 occupies opening 53 and extends into elongate member passage 58 such that barrel portion 41 substantially occupies the larger distal portion 58a of elongate member passage 58. Stem 40 occupies a smaller portion proximal portion 58b of elongate member passage 58. Notches 42 and 44 are aligned with channel 55 and receive locking element 57 positioned in channel 55. Stem 40 is at least partially un-insulated so that a conductive area of stem 40 is positioned at the interface between elongate member passage 58 and relaying chamber 62. This allows lead 26 to be electrically coupled to elongate member 30. The electrical connection between lead 26 and the stem 40 can be maintained by any conventional means known to a person skilled in the art, such as a spring made of a conductive material. Such a spring could be mounted in the relaying chamber 62 where it makes contact with stem 40 of elongate member 30 when elongate member 30 is assembled and seated in handle assembly 50.

In the illustrated embodiment, channel 55 opens along the outside of neck portion 56 and extends approximately three-quarters of the way around neck portion 56. Channel 55 includes through-holes 59 and 61, which are located opposite from one another and open into elongate member passage 58. When handle assembly 50 is viewed in section as shown in FIG. 5, through-holes 59 and 61 are located within channel 55 on the left and right-hand sides of neck portion 56, respectively. Channel 55 begins at first through-hole 59, and extends counterclockwise approximately one-quarter revolution past second through-hole 61, terminating and running out into the outer surface of neck portion 56.

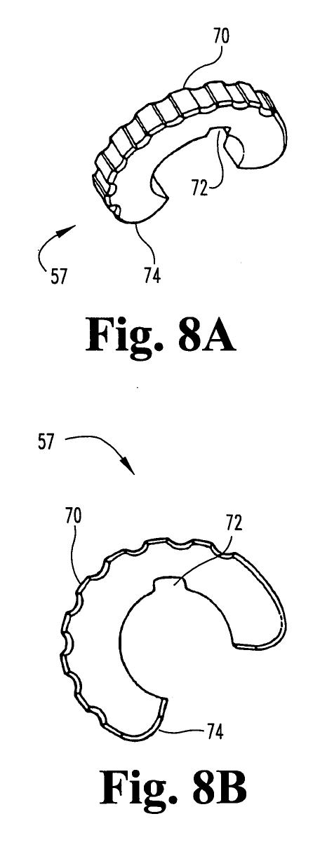

Locking element 57, shown in FIGS. 8A and 8B, is comprised of a substantially flat, semicircular member having a central aperture diameter slightly larger than the inner diameter of channel 55. Locking element 57 includes groove 72 and gripping surface 70, which facilitates rotation of locking element 57 about neck portion 56 in channel 55 by the user. Locking element 57 is adapted to fit within channel 55 and has an outer circumference extending slightly less than three-quarters of the way around neck portion 56, and allows gripping surface to project at least partially from neck portion 56.

Locking element 57 can be manipulated and rotated within channel 55 about a small angular displacement on the order of one-eighth of one rotation. This effectively allows for locking element 57 to be toggled between two positions, which correspond to the locked and unlocked configurations relative to handle assembly 50. When locking element 57 is rotated counterclockwise, no portion of locking element 57 protrudes through through-holes 59 and 61 so that elongate member passage 58 remains clear and unobstructed by locking element 57. In this configuration, groove 72 is aligned with first through-hole 59, and on the other side of channel 55, the end 74 of locking element 57 is located slightly counterclockwise of second through-hole 61. This position corresponds to an unlocked position, which allows removal and insertion of elongate member 30 relative to handle assembly 50. Alternatively, when locking element 57 is rotated clockwise as far as possible, groove 72 is no longer aligned with first through-hole 59, thereby causing a portion of locking element 57 to protrude through first through-hole 59 and obstruct one side portion of elongate member passage 58. Additionally, the end 74 of locking element 57 now protrudes through second through-hole 61, obstructing the other side portion of elongate member passage 58. This position of locking element 57 corresponds to the locked position, where it engages elongate member 30 in handle assembly 50.

In order to join handle assembly 50 to elongate member 30, elongate member 30 is inserted through opening 53 and into passage 58 of handle assembly 50 when locking element 57 is in the unlocked position. If locking element 57 is in the locked position, then side portions of elongate member passage 58 will be obstructed by locking element 57, thereby preventing full insertion of elongate member 30 into handle assembly 50. When barrel portion 41 is fully inserted into elongate member passage 58, the locking element 57 can be rotated so that it engages elongate member 30. The insulated shaft portion 31 overlaps with the insulated outer surface area of handle assembly 50, providing a surgical tool that is entirely insulated proximally of the un-insulated probe end 35.

Once the proximal portion of elongate member 30 has been fully inserted into elongate member passage 58, the proximal stem 41 electrically engages the electrical lead 26 in handle assembly 50. The user may then lock handle assembly 50 to elongate member 30 by rotating locking element 57 to its locked position. As locking element 57 is rotated from its unlocked position to its locked position, elongate member 30 is fixed in place within elongate member passage 58. Portions of locking element 57 protrude through through-holes 59 and 61 into notches 42 and 44 to secure elongate member 30 in position relative to handle assembly 53. The user of surgical tool 21 can use a large amount of force, if necessary, to manipulate surgical tool 21 in order to penetrate tissue and/or bone, without undesired movement of the elongate member 30 relative to handle assembly 51.

While the invention has been illustrated and described in detail in the drawings and foregoing description, the same is to be considered as illustrative and not restrictive in character, and that all changes and modifications that come within the spirit of the invention are desired to be protected.

What is claimed is:

1. A surgical tool for probing bone near neural elements, comprising:
an elongate member extending along a longitudinal axis, said elongate member comprising:
an exposed electrically conductive portion near its distal end for insertion into bone material;
insulated surface area between said distal and a proximal end;
a conductive path between said electrically conductive portion and said proximal end;
a handle assembly having continuously curved surfaces at interfaces with a user's hand, said handle assembly being attachable near said proximal end of said elongate member and comprising:
an electrically insulated surface area;
an electrically conductive area internal to said electrically insulated surface area and engageable with said proximal end of said elongate member;
said handle assembly including a gripping portion having a major dimension at least 50% greater than a minor dimension, said major and minor dimensions being measured orthogonally to said longitudinal axis and to one another; and
an electrical lead extending from said electrically conductive area through said handle assembly.

2. The surgical tool of claim 1, wherein said elongate member is a probe member.

3. The surgical tool of claim 2, wherein said probe member includes a bullet shaped probe end adjacent said distal end.

4. The surgical tool of claim 2, wherein said probe member includes a flattened probe end adjacent said distal end.

5. The surgical tool of claim 2, wherein said probe member includes a probe end that extends transversely to said longitudinal axis.

6. The surgical tool of claim 1, wherein the surgical tool is entirely insulated proximally from said exposed portion when said handle assembly is attached to said elongate member.

7. The surgical tool of claim 1, wherein said elongate member includes an electrically conductive proximal end portion, said proximal end portion fitting inside a receptacle within said handle assembly, said receptacle including an electrical connector receiving said proximal end portion and electrically coupling said elongate member with said lead extending from said receptacle.

8. The surgical tool of claim 1, wherein said handle assembly includes an opening for receiving a non-insulated proximal attachment portion of the elongate member so that said insulated surface area of said handle assembly is in an overlapping arrangement with said insulated area of said elongate member.

9. The surgical tool of claim 8, wherein said handle assembly includes a locking element rotatable around said elongate member from a first position that retains said elongate member in said handle assembly to a second position that allows removal of said elongate member from said handle assembly.

10. The surgical tool of claim 9, wherein said proximal attachment portion includes a notch and said locking element is rotatable about said elongate member for positioning into said notch in said first position and for positioning out of said notch in said second position.

11. The surgical tool of claim 9, wherein said locking element is comprised of an insulated material.

12. The surgical tool of claim 1, wherein said lead extends along said major dimension of said handle assembly and exits said handle assembly at a location distally of said curved surfaces to avoid interfering with the user's hand.

13. A surgical tool for probing bone near neural elements, comprising:
an elongate member extending along a longitudinal axis, said elongate member comprising:
an exposed electrically conductive portion near its distal end for insertion into bone material;
insulated surface area between said distal end and a proximal end;
a conductive path between said electrically conductive portion and said proximal end;
a handle assembly attachable near said proximal end of said elongate member, comprising:
an electrically insulated surface area;
an electrically conductive area internal to said electrically insulated surface area;
said handle assembly having a gripping portion extending along a major dimension transversely to said longitudinal axis, said insulated surface area of said handle assembly being positioned in overlapping relation with said insulated surface area of said elongate member when said elongate member is attached to said handle assembly; and
an electrical lead electrically engaging said proximal end of said elongate member in said handle assembly.

14. The surgical tool of claim 13, wherein said elongate member includes an electrically conductive proximal end portion, said proximal end portion fitting inside a receptacle within said handle assembly, said receptacle including an electrical connector receiving said proximal end portion and electrically coupling said elongate member with said lead extending from said receptacle.

15. The surgical tool of claim 13, wherein said handle assembly includes continuously curved surfaces along said gripping portion at interfaces with a user's hand to provide an anatomical fit therewith.

16. The surgical tool of claim 13, wherein said handle assembly includes a locking element rotatable around said elongate member from a first position that retains said elongate member in said handle assembly to a second position that allows removal of said elongate member from said handle assembly.

17. The surgical tool of claim 16, wherein said elongate member includes a proximal attaching portion positionable in said handle assembly, said attaching portion including at least one notch and said locking element is rotatable about said longitudinal axis of said elongate member for positioning into said at least one notch in said first position and for positioning out of said at least one notch in said second position.

18. The surgical tool of claim 13, wherein said electrical lead extends from said proximal end of said elongate member internally of said insulated surface area of said handle assembly and along said major dimension of said gripping portion.

19. A surgical tool for probing bone near neural elements, comprising:
an elongate member extending along a longitudinal axis, said elongate member comprising:
an exposed, electrically conductive portion near a distal end;
a proximal portion;
insulated surface area extending about a conductive path between said conductive portion and said proximal portion;
a handle assembly, comprising:
an electrically insulated surface area;
an electrically conductive area internal to said electrically insulated surface area;
an opening for receiving said proximal portion of said elongate member in electrical engagement with said electrically conductive area and with said insulated surface area of said handle assembly in an overlapping arrangement with said insulated surface area of said elongate member; and
a gripping portion extending along a major dimension and an electrical lead extending from said electrically conductive area along said major dimension internally of said electrically insulated surface area.

20. The surgical tool of claim 19, further comprising a locking element rotatable around said elongate member from a first position that retains said elongate member in said handle assembly to a second position that allows removal of said elongate member from said handle assembly.

21. The surgical tool of claim 19, wherein said handle assembly includes a receptacle including an electrical connector for electrically engaging said proximal portion of said elongate member and electrically coupling said elongate member with said lead.

22. The surgical tool of claim 19, wherein said proximal portion is non-rotatably received inside said opening of said handle assembly.

23. The surgical tool of claim 19, wherein said major dimension of said gripping portion is at least 50% greater than a minor dimension, said major and minor dimensions being measured orthogonally to said longitudinal axis and to one another.

24. The surgical tool of claim 19, wherein said gripping portion of said handle assembly includes continuously curved surfaces providing an anatomical fit at interfaces with a user's hand along said major dimension.
